(19) United States
(12) Patent Application Publication
(10) Pub. No.: US 2010/0112657 A1
(43) Pub. Date: May 6, 2010

(54) NUCLEIC ACIDS AND CONSTRUCTS FOR INCREASING GALACTOSE CATABOLISM AND METHODS THEREFOR

(75) Inventors: Byung Jo YU, Hwaseong-si (KR); Jae Chan PARK, Yongin-si (KR); Hyun Min KOO, Seoul (KR); Yong Su JIN, Suwon-si (KR); Ki Sung LEE, Suwon-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 12/435,697

(22) Filed: May 5, 2009

(30) Foreign Application Priority Data
Oct. 30, 2008 (KR) 10-2008-0107277

(51) Int. Cl. C12P 7/06; C07H 21/00; C07K 14/00; C12N 1/16; C12N 1/00; C12N 15/74

(52) U.S. Cl. 435/161; 536/23.1; 530/350; 435/320.1; 435/254.2; 435/254.21; 435/254.22; 435/254.23; 435/471

(57) ABSTRACT

Provided are a recombinant gene associated with increased galactose catabolism, and a recombinant vector and microorganism including the gene. Also disclosed are a method of producing ethanol from a galactose-containing carbon source by culturing the microorganism including the gene in a galactose-containing carbon source such that ethanol is produced, and a method of screening a gene in yeast resulting in increased galactose catabolism when overexpressed.

### CROSS-REFERENCE TO RELATED APPLICATION

This application claims the benefit of Korean Patent Application No. 10-2008-0107277, filed Oct. 30, 2008, hereby incorporated by reference in its entirety.

### BACKGROUND

1. Field

This application relates to a recombinant gene associated with increased galactose catabolism, a recombinant vector and microorganism containing the same, and to methods of making and using these compositions. More particularly, this application relates to a recombinant gene repressing expression of a gene involved in galactose catabolism, in which all or a part of a repression domain is inactivated.

2. Description of the Related Art

With the globally increasing concern about exhaustion of resources and pollution of the environment due to overuse of fossil fuels, development of new and renewable alternative energy sources for stable and continuous production of energy is being considered. Among such alternative energy resources under development, technology for producing energy from biomass has been receiving considerable attention.

In recent times, there has been considerable interest in the prospect of using algae as a source of biomass. An advantage of algae is its abundance and rapid growth. Also, since algae consume carbon dioxide and exhaust oxygen for growth, they offer a potential solution to both energy production and pollution concerns. However, algae have not yet been produced on a large scale for use in a variety of applications. Additionally, hydrolysates of biomass derived from algae contain a large amount of galactose. Accordingly, effective use of the abundant galactose in such hydrolysates is the first step toward developing a biological process for converting hydrolysates of algae-derived biomass into useful materials by fermenting organisms, such as yeast.

However, although galactose can be catabolized by naturally occurring microorganisms, such as yeast, the uptake and metabolic utilization rate of galactose is much lower than that of glucose.

### SUMMARY

Disclosed herein is a novel recombinant gene increasing galactose catabolism when overexpressed, and a method of increasing volumetric productivity of ethanol from a carbon source containing galactose by using yeast, or other fermenting microorganism, transformed with the recombinant gene.

Disclosed herein is an isolated polynucleotide. In an embodiment, the isolated polynucleotide has a nucleotide sequence encoding a recombinant polypeptide, the amino acid sequence of which consists of SEQ ID NO:2.

Also disclosed herein is a recombinant polypeptide. In an embodiment, the isolated polypeptide has the amino acid sequence of SEQ ID NO:2.

In another embodiment, a recombinant vector including the isolated polynucleotide is disclosed.

In another embodiment, a recombinant microorganism including the isolated polynucleotide is disclosed.

Also disclosed is a method of producing the recombinant microorganism. In an embodiment, the method includes transforming a microorganism with the recombinant vector including the isolated polynucleotide.

Also disclosed herein is a method of producing ethanol from a galactose-containing carbon source. In an embodiment, the method includes culturing a recombinant microorganism disclosed herein in a galactose-containing carbon source such that ethanol is produced.

Also disclosed herein is a method of screening yeast genes to select a yeast gene for increasing galactose catabolism when overexpressed. In an embodiment, the method includes: constructing a genomic DNA library of the yeast using a multi-copy plasmid containing trp; transforming the yeast using the constructed genomic DNA library, and constructing a library of the transformed yeast in which all yeast genes are overexpressed; culturing the constructed transformed yeast library in a medium containing only galactose as a carbon source, and screening the transformed yeast having increased galactose utilization, which form big colonies through serial subculture; and isolating the plasmid from the screened transformed yeast, and identifying a yeast genomic sequence inserted into the isolated plasmid.

These and other embodiments, advantages and features of the invention become clear when detailed description and examples are provided in subsequent sections.

### BRIEF DESCRIPTION OF THE DRAWINGS

Exemplary embodiments are described in further detail below with reference to the accompanying drawings. It should be understood that various aspects of the drawings may have been exaggerated for clarity.

FIG. 1 is a schematic diagram of TUP1 protein, with the dotted line bisecting the sequence indicating the truncation point after amino acid 284 for the mutant described in Example 1;

FIG. 2 is a schematic diagram of the truncated TUP1 protein of Example 1 in which the C-terminal repression domain has been deleted;

FIG. 3 shows a method of screening genes resulting in increased galactose catabolism;

FIG. 4 is an enlarged genetic map of the region of yeast chromosome III containing the TUP1 gene (261594˜263396);

FIG. 5 is a cleavage map of plasmid pRS424 used in Example 1 to construct the genomic library;

FIGS. 6 and 7 are graphs showing ethanol production resulting from culturing S. cerevisie in minimal media containing a sugar mixture (containing 2% glucose and 2% galactose) as well as OD₆₀₀ and concentration of glucose and galactose remaining (expressed as g/L) as a function of time (FIG. 6: Wild-type strain; FIG. 7: truncated TUP1-overexpressing stain);

FIGS. 8 and 9 are graphs showing ethanol production resulting from culturing S. cerevisie in minimal media containing 4% galactose as well as OD₆₀₀ and galactose (in g/L) remaining as a function of time (FIG. 8: Wild-type stain; FIG. 9: truncated TUP1-overexpressing stain);

FIGS. 10 and 11 are graphs showing ethanol production resulting from culturing S. cerevisie in minimal media containing 10% galactose as well as OD₆₀₀ and the galactose remaining as a function of time (FIG. 10: Wild-type strain; FIG. 11: truncated TUP1-overexpressing stain); and

FIG. 12 is a graph showing ethanol production resulting from culturing four different S. cerevisie strains (wild-type; wild-type TUP1 overexpressing; truncated TUP1 overexpressing; and TUP1 knockout) in minimal media containing 10% galactose as a function of time.

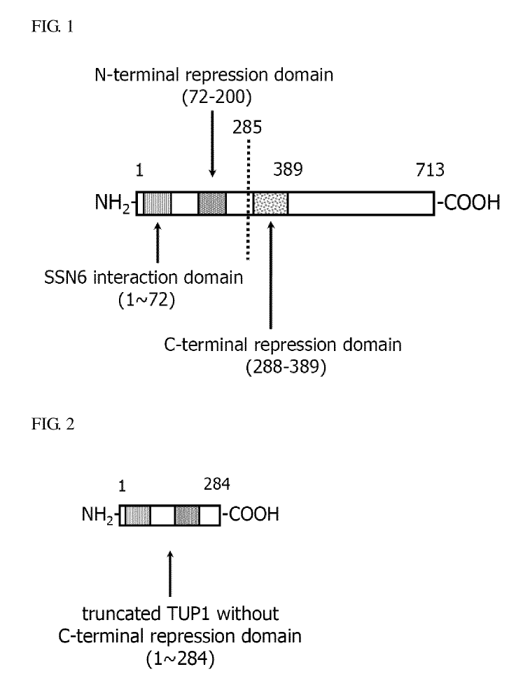

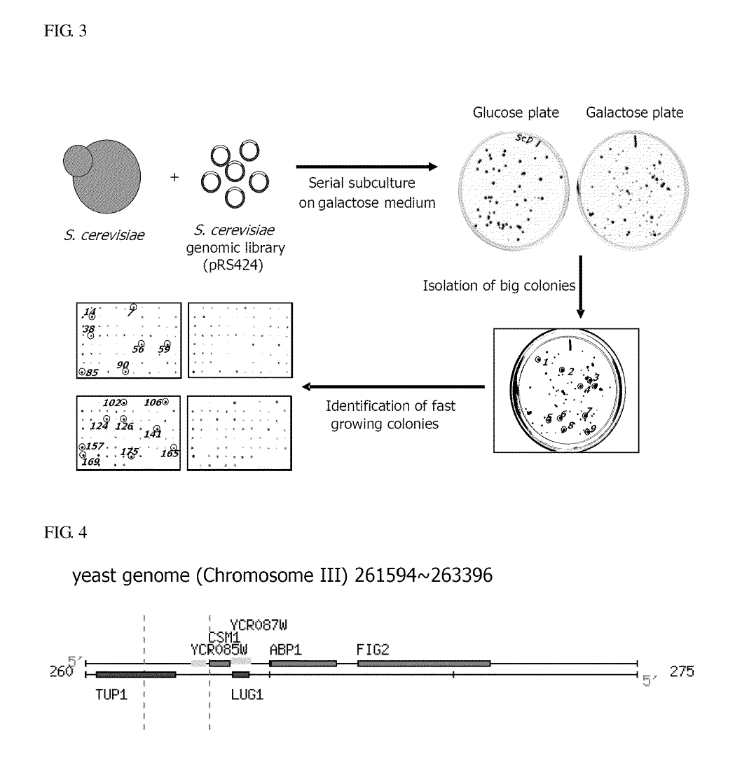

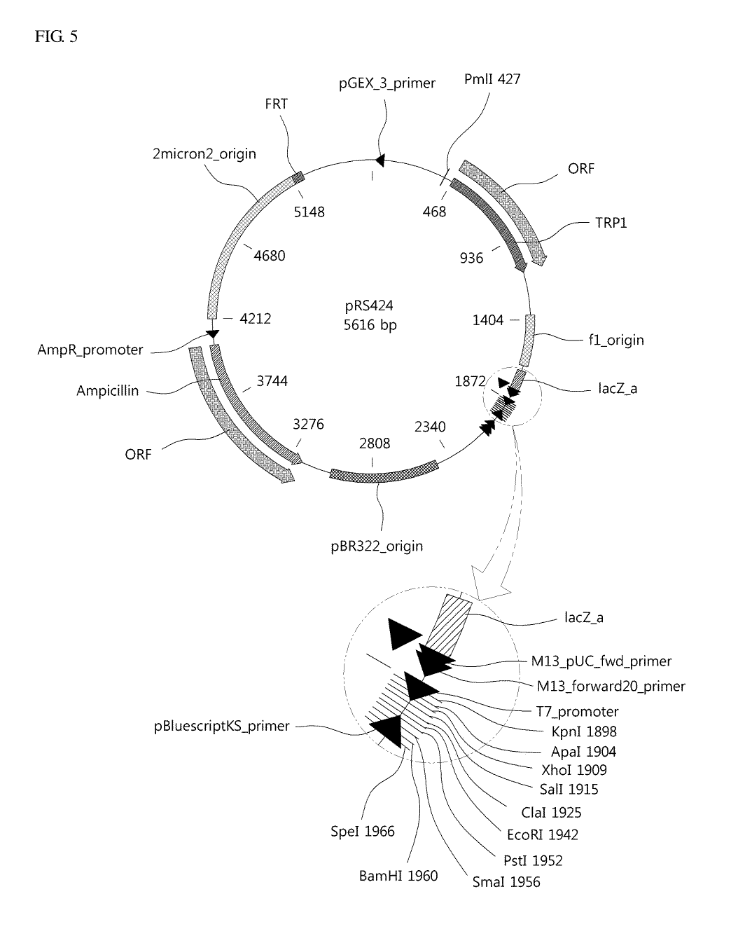

### DETAILED DESCRIPTION

Hereinafter, the inventive concept will now be described more fully with reference to exemplary embodiments and the accompanying drawings. However, it should be understood that the inventive concept is not limited to the described exemplary embodiments and may be embodied in various modifications and changes.

### 1. Recombinant Gene and Recombinant Protein Encoded Therein

A novel recombinant repressor gene is provided. In an embodiment of the recombinant repressor gene, the repressor gene is truncated such that all or a portion of a repression domain is deleted from the encoded protein. The wild-type repressor protein represses expression of a galactose-catabolizing gene.

Galactose is an aldohexose, molecular formula C₆H₁₂O₆, which is converted into galactose-1-phosphate and then glucose-1-phosphate in many organisms. In some microorganisms, e.g., yeast, the glucose-1-phosphate can be catabolized into ethanol by fermentation. Herein, the concentration of ethanol produced per galactose consumption time is denoted as the “volumetric productivity of ethanol”.

Galactose-catabolizing genes in yeast, i.e., genes involved in galactose uptake and metabolism, include gal2, gal1, gal7, gal10, and gal5 (pgm1 or pgm2).

The term ‘galactose catabolism’ refers to the metabolic degradation of galactose. The level of galactose catabolism is often expressed as the ‘galactose utilization rate’.

A gene encoding a protein comprising a repression domain (‘repressor gene’) may include tup1, gal4, gal3, gal80, gal6, mig1, and ssn6. By genetic manipulation of these repressor genes, expression of the galactose-catabolizing gene may not be suppressed. For example, expression of a galactose-catabolizing gene may be increased by deleting or modifying a repression domain that functions as an activation site of the repressor protein.

In one example, the repressor gene can be the gene encoding TUP1 protein. In some embodiments, the repressor gene can be a yeast gene. Specifically, the repressor gene can be from S. cerevisiae.

The TUP1 protein is known to function as a general transcriptional co-repressor in yeast. Among the genes which TUP1 regulates are genes involved in galactose catabolism. In particular, Saccharomyces cerevisiae TUP1 forms a complex with CYS8 (SSN6), which represses transcription of genes regulated by glucose, oxygen, and DNA damage.

While neither TUP1 nor CYS8(SSN6) binds directly to DNA, each functions as an element of a co-repressor through interaction with various DNA-binding proteins such as α 2, Mig1, Rox1, or a1. For example, the TUP1 protein can bind to SSN6 protein thereby forming a complex that suppresses expression of a galactose-catabolizing gene, guided by the DNA binding protein Mig1.

Therefore, when a part of the repression domain in TUP1 is inactivated, for example by deletion, expression of the galactose-catabolizing genes may not be suppressed. For convenience, a TUP1 protein in which a part of the expression repression domain is deleted is called a “truncated TUP1 protein”.

Accordingly, without being bound by theory, it is believed that when the gene encoding truncated TUP1 protein is overexpressed, the repression mechanism does not operate properly, resulting in expression of the galactose-catabolizing genes.

All or part of the repression domain of the repressor gene may be deleted. If at least two repression domains are present in the repressor gene, then only one or both of them may be deleted.

In an exemplary embodiment, the repression domain may be a C-terminal repression domain. In an embodiment, the truncated gene can be a gene encoding TUP1 protein in which all or part of its C-terminal repression domain is deleted.

Herein, a “C-terminal domain” refers to a domain including the carboxylic acid terminus of the protein, corresponding in the gene to the amino acid sequence encoded by the nucleic acid sequence located at the 3′ end of the protein-coding region of the gene.

The C-terminal domain of a repressor protein may include the final 300 amino acids counting from the C-terminus of the polypeptide, or the final ⅓ of the polypeptide counting from the C-terminus of the polypetide. The “C-terminal domain” of a polypeptide is not shorter than 3 amino acids and not longer than 350 amino acids and may include 5, 10, 20, 25, 50, 100, or 200 amino acids.

The C-terminal half of TUP1 protein contains six repeats of a 43-amino acid sequence motif rich in aspartate and tryptophan, known as WD-40 or β-transducin repeats. WD-40 repeats have been identified in many proteins and are known to play a role in protein——protein interactions.

Thus, the portion of the C-terminal repression domain deleted from the truncated TUP1 may include one or more WD-40 repeats.

In one example, the truncated gene may encode TUP1 protein in which a portion of the C-terminal repression domain found at positions 288-389 in the amino-acid sequence is deleted. Herein, a gene encoding a truncated TUP1 protein in which a portion of positions 288-389 in the amino-acid sequence is deleted is referred to as a “truncated tup1” gene.

In one example, the truncated TUP1 protein can be truncated after amino-acid 284. The isolated polypeptide of such a truncated TUP1 protein has the amino acid sequence in SEQ ID NO:2. The truncated TUP1 protein with the amino acid sequence of SEQ ID NO:2 includes the SSN6 interaction domain and the N-terminal repression domain at amino acid positions 72 to 200. In another example, the recombinant TUP1 protein is encoded by the polynucleotide sequence of SEQ ID NO:1.

In another exemplary embodiment, a recombinant TUP1 protein in which a part of the C-terminal repression domain is truncated is provided. In this embodiment, the recombinant TUP1 protein has a partially deleted C-terminal repression domain, so that TUP1 becomes inactive in repressing expression of glucose-catabolizing genes, and instead permits expression of galactose-catabolizing genes.

Isolated polynucleotides encoding the recombinant repressor proteins are also provided.

“Isolated,” when used to describe the various polypeptides or polynucleotides disclosed herein, means a polypeptide or polynucleotide that has been identified and separated and/or recovered from a component of its natural environment. The term also embraces recombinant polynucleotides and polypeptides and chemically synthesized polynucleotides and polypeptides.

In an embodiment, the isolated polynucleotide consists of a nucleotide sequence encoding a recombinant protein, the amino acid sequence of which consists of SEQ ID NO:2.

In another embodiment, the isolated polynucleotide consists of the nucleotide sequence of SEQ ID NO:1.

### 2. Recombinant Vector and Recombinant Microorganism

A recombinant vector including the isolated polynucleotide encoding a TUP1 protein in which all or part of one of the repression domains is deleted is provided. In an embodiment, the recombinant vector comprises an isolated polynucleotide encoding a truncated TUP1 protein having the amino acid sequence of SEQ ID NO:2.

The term “vector” refers to a nucleic acid molecule capable of transporting another nucleic acid to which it has been linked. Available vectors may include bacteria, plasmids, phages, cosmids, episomes, viruses, and insertable DNA fragments (fragments able to be inserted into a host cell genome by homologous recombination).

The term “plasmid” refers to a circular, extra-chromosomal, double-stranded DNA molecule typically capable of autonomous replication within a suitable host and into which a foreign DNA fragment can be inserted. Further, the term “virus vector” refers to a vector in which foreign DNA has been inserted into a virus genome for delivery into cells.

Herein, a vector directing expression of a gene encoding a target protein operably linked thereto is called an “expression vector.” Generally, in recombinant DNA technology, a plasmid is used as an expression vector, and thus the term “plasmid” may be interchangeably used with the term “expression vector.” However, it should be clear that expression vectors also include other types of vectors exhibiting the same function, for example, a virus vector.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter) or a ribosome binding site is operably linked to a coding sequence if it is positioned so as to facilitate translation. Coding sequences can be operably linked to regulatory sequences in a sense or antisense orientation.

In some embodiments, the expression vector can introduce and express a specific gene 1 in yeast. Examples of such expression vectors include vector II micron, pBM272, pBR322-6, pBR322-8, pCS19, pDW227, pDW229, pDW232, pEMBLYe23, pEMBLYe24, pEMBLYi21, pEMBLYi22, pEMBLYi32, pEMBLYr25, pFL2, pFL26, pFL34, pFL35, pFL36, pFL38, pFL39, pFL40, pFL44L, pFL44S, pFL45L, pFL45S, pFL46L, pFL46S, pFL59, pFL59+, pFL64−, pFL64+, pG6, pG63, pGAD10, pGAD424, pGBT9, pGK12, pJRD171, pKD1, pNKY2003, pNKY3, pNN414, pON163, pON3, pPM668, pRAJ275, pRS200, pRS303, pRS304, pRS305, pRS306, pRS313, pRS314, pRS315, pRS316, pRS403, pRS404, pRS405, pRS406, pRS413, pRS414, pRS415, pRS416, pRS423, pRS424, pRS425, pRS426, pRSS56, pSG424, pSKS104, pSKS105, pSKS106, pSZ62, pSZ62, pUC-URA3, pUT332, pYAC2, pYAC3, pYAC4, pYAC5, pYAC55, pYACneo, pYAC-RC, pYES2, pYESHisA, pYESHisB, pYESHisC, pYEUra3, rpSE937, YCp50, YCpGAL0, YCpGAL1, YCplac111, YCplac22, YCplac33, YDp-H, YDp-K, YDp-L, YDp-U, YDp-W, YEp13, YEp213, YEp24, YEp351, YEp352, YEp353, YEp354, YEp355, YEp356, YEp356R, YEp357, YEp357R, YEp358, YEp358R, YEplac112, YEplac181, YEplac195, YIp30, YIp31, YIp351, YIp352, YIp353, YIp354, YIp355, YIp356, YIp356R, YIp357, YIp357R, YIp358, YIp358R, YIp5, YIplac128, YIplac204, YIplac211, YRp12, YRp17, YRp7, pAL19, paR3, pBG1, pDBlet, pDB248X, pEA500, pFL20, pIRT2, pIRT2U, pIRT2-CAN1, pJK148, pJK210, pON163, pNPT/ADE1-3, pSP1, pSP2, pSP3, pSP4, pUR18, pUR19, pZA57, pWH5, pART1, pCHY21, pEVP11, REP1, REP3, REP4, REP41, REP42, REP81, REP82, RIP, REP3X, REP4X, REP41X, REP81X, REP42X, REP82X, RIP3X/s, RIP4X/s, pYZ1N, pYZ41N, pYZ81N, pSLF101, pSLF102, pSLF104, pSM1/2, p2UG, pART1/N795, and pYGT.

In some embodiments, the isolated polynucleotide is inserted into a multicopy plasmid to supply multiple copies of the inserted gene for high expression of the truncated TUP1 protein. Alternatively, the isolated polynucleotide is inserted into a low-copy plasmid containing a strong promoter to achieve high expression of the protein. In one example, the plasmid is plasmid pRS424, having the cleavage map shown in FIG. 5.

The vector may be introduced into a host cell, and produce a protein or peptide such as a fusion protein or peptide encoded by the genes mentioned above. In some cases, the vector may contain a promoter recognized by the host cell. By “promoter” is meant minimal sequence sufficient to direct transcription. Also included are those promoter elements which are sufficient to render promoter-dependent gene expression controllable for cell-type specific or inducible by external signals or agents; such elements may be located in the 5′ or 3′ regions of the gene. Both constitutive and inducible promoters are included (see e.g., Bitter et al. (1987) Methods in Enzymology 153: 516-544).

The promoter sequence may originate from a prokaryote, a eukaryote, or a virus. Yeast-compatible promoters include GAPDH, PGK, ADH, PHO5, GAL1 and GAL10. Selecting a yeast-compatible promoter with a suitable promoter activity for the desired level of expression of a gene in yeast is within the skill of the ordinary artisan.

The vector may have an additional expression control sequence. “Control sequences” refers to DNA sequences necessary for the expression of an operably linked coding sequence in a particular host organism. The control sequence may be a Shine-Dalgarno sequence. For example, the Shine-Dalgarno sequence can be from the replicase gene of phage MS-2 or from the cII gene of bacteriophage λ. Moreover, the vector may have an appropriate marker to screen the transformed host cell. The transformation of a host may be accomplished by any one of a variety of techniques well known in the art.

A recombinant microorganism comprising the isolated polynucleotide encoding a truncated TUP1 protein is also provided. In an embodiment, the recombinant microorganism comprises an isolated polynucleotide encoding the truncated TUP1 protein having the amino acid sequence of SEQ ID NO:2.

The microorganism may be any known in the art, such as a bacteria, a fungus, or a yeast.

In some embodiments, the microorganism is a yeast. The yeast is selected from the genus Saccharomyces, the genus Pachysolen, the genus Clavispora, the genus Kluyveromyces, the genus Debaryomyces, the genus Schwanniomyces, the genus Candida, the genus Pichia, or the genus Dekkera.

Without being bound by theory, it is believed that in such a recombinant yeast strain, the repression activity of wild-type TUP1 protein is inhibited by the gene encoding the truncated TUP1 protein, and thus repression of expression of a galactose-catabolizing gene does not operate properly. As a result, expression of the galactose-catabolizing gene is stimulated, and then galactose may be rapidly converted to ethanol in a medium containing as the carbon source either a mixture of glucose and galactose or only galactose. Thus, the recombinant yeast strain can exhibit improved volumetric productivity of ethanol from a process of culturing the recombinant yeast strain in a galactose-containing medium.

The recombinant microorganism may increase ethanol volumetric productivity at least about 30%, or at least about 50% over the ethanol volumetric productivity of the wild-type microorganism.

The recombinant microorganism can be produced by transforming the parent microorganism with a recombinant vector disclosed herein. Transformation of the microorganism is conducted by any transformation method known to a person of ordinary skill in the art. For example, transformation of a yeast with the recombinant vector may be conducted by the method of Ito, H., et al. (J. Bacteriol.(1983) 153, 163-168).

In some embodiments in order to transform S. cerevisiae CEN.PK2-1D with a vector containing a heterogeneous gene, a spheroplast transformation kit (Bio 101, Vista, Calif.) is used. In such an embodiment, the transformed strain may be cultured in yeast synthetic complete (YSC) medium containing 20g/l of glucose, and then continuously cultured in YSC medium containing 4% galactose. Afterward, strains with an improved galactose utilization rate may be screened on 4% galactose-containing YSC solid medium.

In one embodiment, a recombinant microorganism deposited as Accession No. KCTC 11387 BP is provided.

The deposited recombinant microorganism, determined by screening to have an excellent galactose utilization rate, was deposited under the name of Saccharomyces cerevisiae CEN.PK2-1D/pRS424-truncated TUP1 on Sept. 4, 2008 to the Gene bank of the Korea Research Institute of Bioscience and Biotechnology (Yuseung-gu, Daejeon, Korea) with Accession No. KCTC 11387 BP.

### 3. Method of Producing Ethanol

A method of producing ethanol from a galactose-containing carbon source is also provided. In an embodiment, the method comprises culturing a recombinant microorganism disclosed herein in a galactose-containing carbon source such that ethanol is produced. The method can further comprise recovering the ethanol.

Herein, when the gene is overexpressed, volumetric productivity of ethanol is increased.

The galactose-containing carbon source may contain a mixture of galactose and glucose, or only galactose. The mixed ratio of the galactose to glucose in the medium is not particularly limited, but the medium may contain at least about 4.0% galactose.

As shown below in the Examples, culturing the recombinant microorganism that overexpresses the truncated TUP1 gene in a medium containing either a mixture of glucose and galactose, or only galactose, as a carbon source, yields greatly increased volumetric productivity of ethanol, compared to the parent microorganism.

The galactose-containing carbon source may be a hydrolysate of algae biomass.

The kind of algae is not particularly limited and may include red algae (e.g., Porphyra yezoensis Ueda), brown algae (e.g., the Laminariaceae family, Undaria pinnatifida and Hizikia fusiforme), and green algae (e.g., Enteromorpha genus).

The red algae may include Gelidium amansii, Gracilaria verrucosa, Bangia atropurpurea, Porphyra suborbiculata, Porphyra yezoensis, Galaxaura falcate, Scinaia japonica, Gelidium divaricatum, Gelidium pacificum, Lithophylum okamurae, Lithothammion cystocarpideum, Amphiroa anceps, Amphiroa beauvoisii, Corallina officinalis, Corallina pilulifera, Marginisporum aberrans, Carpopeltis prolifera, Grateloupia filicina, Grateloupia elliptica, Grateloupia lanceolanta, Grateloupia turtuturu, Phacelocarpus japonicus, Gloiopeltis furcata, Hypnea charoides, Hypnea japonitca, Hypnea saidana, Chondrus cripspus, Chondracanthus tenellus, Gracilaria textorii, Lomentaria catenata, Heterosiphonia japonica, Chondria crassicaulis, and Symphyocladia latiuscula.

A method of producing ethanol from algae biomass may be conducted by any method known in the art. For example, ethanol may be produced from red algae biomass by direct saccharification, in which the red algae are directly saccharified, or by indirect saccharification in which agar or cellulose is extracted from the red algae and then saccharified to obtain galactose or glucose. The saccharification may be performed by enzyme hydrolysis using galactocidase, or acid hydrolysis using a catalyst for acid hydrolysis. Then, ethanol may be produced by fermentation using any microorganism.

When ethanol is produced from algae biomass, which is abundant in nature, resource supply and demand can be stable and no pretreatment processes are necessary. Thus, very high production efficiency may be obtained.

### 4. Method of Screening Genes

A method of screening for genes causing increased catabolic utilization of galactose in yeast when the genes are overexpressed is provided. In an embodiment, the method includes the following steps: constructing a yeast genomic DNA library using a trp-containing multi-copy plasmid; transforming yeast with the genomic DNA library; preparing a library of transformed yeast in which the genes inserted into the multi-copy plasmid are overexpressed; culturing the transformed yeast library in a medium containing only galactose as a carbon source, and screening the transformed yeast for colonies exhibiting increased galactose utilization, by identifying fast-growing colonies by serial subculture; isolating the plasmid from the screened yeast; and identifying a yeast genomic sequence inserted into the isolated plasmid.

FIG. 3 illustrates one embodiment of a method of screening for genes yielding increased galactose utilization. The gene screening method will be described in detail with reference to FIG. 3.

In an embodiment of the method, the yeast may be S. cerevisiae CEN.PK2-1D, and the multi-copy plasmid may be pRS424.

In an embodiment of the method, the yeast genomic DNA library can be prepared by cutting S. cerevisiae CEN.PK2-1D genomic DNA with a restriction enzyme, ligating a yeast DNA fragment into the multi-copy plasmid (pRS424), and amplifying the recombinant plasmid in E. coli.

In an embodiment of the method, the transformed yeast library may be prepared by the method described by Ito et al. (J.bacteriol. (1983) 153, 163-168).

Then, the transformed yeast are selected for those with increased galactose utilization. Such colonies exhibit faster growth as evidenced by the formation of big colonies in serial subculture. Afterward, the inserted yeast genomic sequence in the selected transformed yeast is identified. In some embodiments, the genomic sequence may be analyzed using a gel documentation (gel doc) device.

In an embodiment, the gene screening method may further include: detecting the location of the inserted gene on the yeast genome by comparing the base sequence of the yeast genome with a predetermined length of genomic sequence present at each end of the gene inserted into the plasmid to identify the overexpressed gene; or re-transforming a yeast with the plasmid containing the identified gene to confirm an increase in galactose catabolism is caused by overexpression of the gene.

In one embodiment, the gene increasing galactose catabolism upon overexpression encodes a TUP1 protein in which at least a part of the C-terminal repression domain is deleted, as described above.

The following examples further illustrate the invention but, of course, should not be construed as in any way as limiting its scope.

### PREPARATION EXAMPLE 1

The recombinant TUP1 gene was selected using the procedures schematically described in FIG. 3.

A yeast genomic DNA library was constructed by ligating size-selected S. cerevisiae genomic DNA fragments into a multi-copy plasmid (pRS424). Subsequently, competent E. coli cells were transformed with the recombinant multicopy plasmids to create the genomic library.

S. cerevisiae CEN.PK2-1D (MATalpha; ura3-52; trp1-289; leu2-3_{—}112; his3 D1; MAL2-8C; SUC2) was then transformed with the S. cerevisiae genomic library to construct a transformed yeast library in which all the S. cerevisiae genes are overexpressed.

The transformed yeast library was cultured in a medium containing only galactose as a carbon source. The cultured transformed yeast were then screened by serial subculturing to select those yeast strains exhibiting increased galactose utilization. Transformed yeast exhibiting increased galactose utilization will grow more quickly on a medium containing only galactose as a carbon source than yeast with normal galactose utilization and therefore will form big colonies. Plasmids were isolated from the yeast exhibiting increased galactose utilization to permit determination of the yeast genomic sequence inserted into the plasmid by sequencing.

The location of the yeast genomic sequence insert on the yeast genome was determined by comparing the base sequence of the yeast genome with a predetermined length of genomic sequence present at each end of a gene inserted into the plasmid to identify the gene;

A plasmid with an inserted gene encoding TUP1 protein (3rd chromosome 261594-263396) in which the part of the C-terminal repression domain beyond amino acid 284 (the polypeptide of SEQ ID NO:2) was identified in general accordance with the above procedures.

S. cerevisie CEN.PK2-1D was then transformed with the multi-copy plasmid encoding the truncated TUP1 protein containing only amino acids 1-284 using a spheroplast transformation kit (Bio 101, Vista, Calif.) in order to confirm that overexpression of the gene resulted in an increase in galactose catabolism. The transformed strain was cultured in yeast synthetic complete (YSC) medium containing 20 g/l of glucose, and as much amino acids and nucleotides as needed were provided.

### PREPARATIVE EXAMPLE 2

A strain of Pichia sp is transformed with a multi-copy plasmid encoding the truncated TUP1 protein. The transformed strain is cultured in YSC medium containing 20 g/l of glucose, and other necessary nutrients.

### PREPARATIVE EXAMPLE 3

A strain of Kluyveromyces sp is transformed with a multi-copy plasmid encoding the truncated TUP1 protein. The transformed strain is cultured in YSC medium containing 20 g/l of glucose, and other necessary nutrients.

### EXAMPLE 1

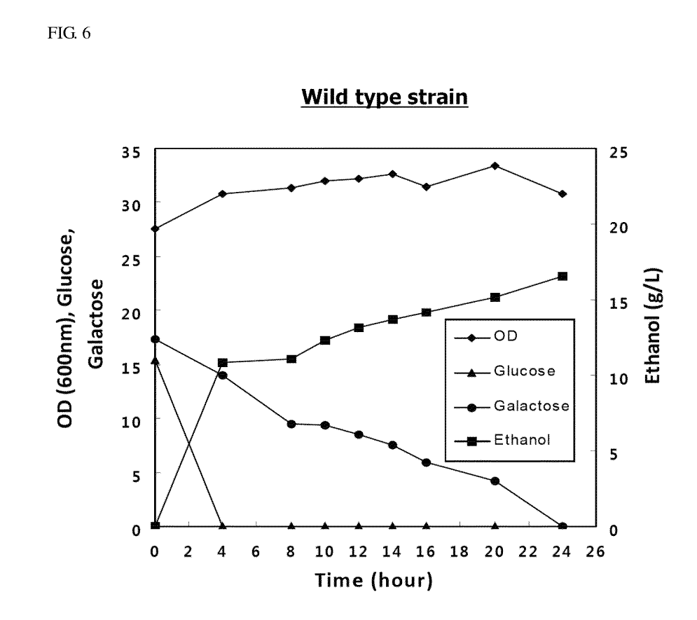

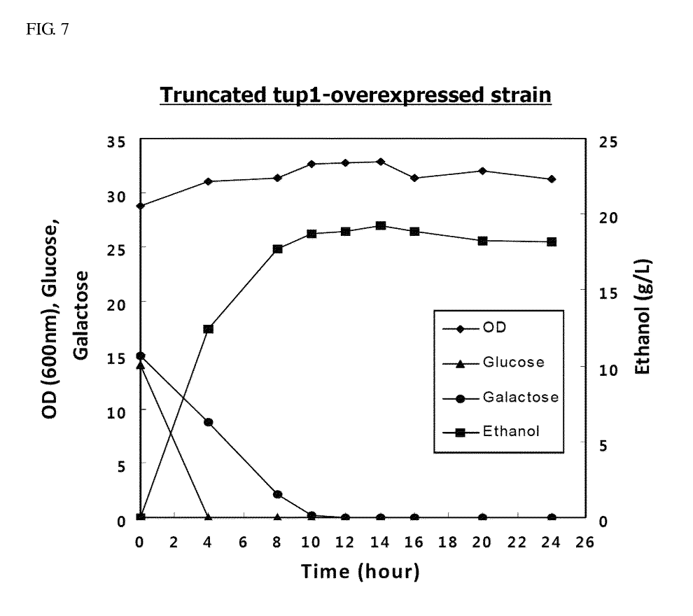

A culture of the wild type yeast strain (wild type) containing an empty vector and a culture of the recombinant yeast strain transformed with the gene encoding truncated TUP1 protein (truncated tup1) prepared in Preparation Example 1 were inoculated to achieve an initial OD₆₀₀=25 and then cultured in minimal medium containing a mixture of 2% each of glucose and galactose. Subsequently, galactose utilization and volumetric productivity of ethanol of each culture were determined. The results are shown in FIGS. 6 and 7, for the wild-type strain and the recombinant strain, respectively, and in Table 1.

TABLE 1

| | Wild type | Truncated Tup1 |
|---|---|---|
| Galactose consumption time | 24 h | 10 h |
| Ethanol concentration | 16.5 g/l | 18.9 g/l |
| Volumetric productivity | 0.69 g/l h | 1.90 g/l h |

The results in FIGS. 6 and 7 and in Table 1 show that the galactose utilization rate is significantly increased in the recombinant yeast strain transformed with the gene encoding truncated TUP1 protein, compared to the wild type strain.

Particularly, while the time to consume 2% galactose by the recombinant yeast strain transformed with the gene encoding truncated TUP1 protein was about 10 hours, the time for the wild type strains was about 24 hours. In addition, while the final ethanol concentration produced by the yeast strain transformed with the gene encoding truncated TUP1 protein was 18.9 g/L, that produced by the wild type strains was 16.5 g/L.

### EXAMPLE 2

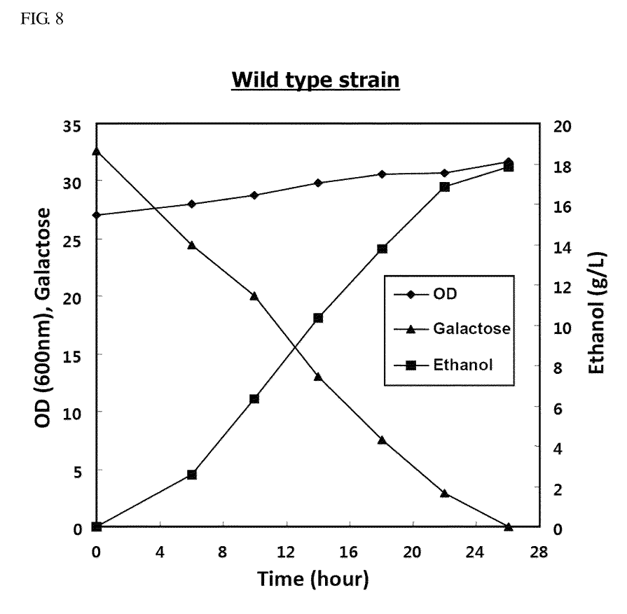

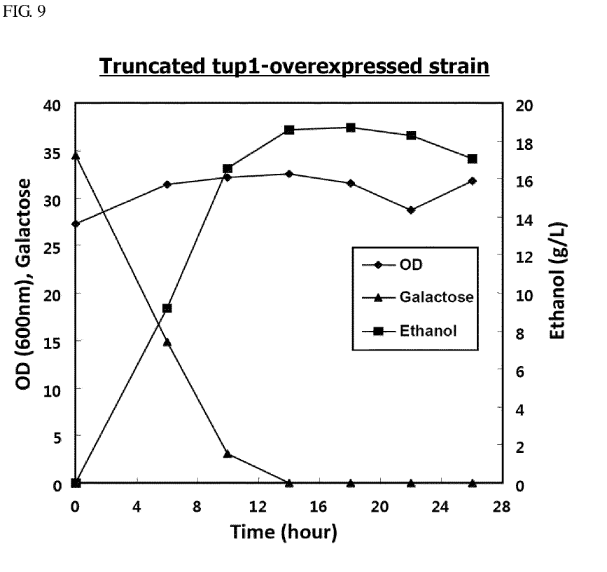

The wild type yeast strain (wild type) was compared to the recombinant yeast stain transformed with the gene encoding truncated TUP1 protein (truncated TUP1) prepared in Preparation Example 1 with respect to galactose utilization and volumetric productivity of ethanol by the same method as described in Example 1, except that the yeast strains were cultured in minimal medium containing only 4% galactose as a carbon source. The results are shown in FIGS. 8 and 9, for wild-type and recombinant yeast strains, respectively, and Table 2.

TABLE 2

| | Wild type | Truncated tup1 |
|---|---|---|
| Galactose consumption time | 26 h | 14 h |
| Ethanol concentration | 17.8 g/l | 18.7 g/l |
| Volumetric productivity | 0.68 g/lh | 1.33 g/lh |

The results show that the galactose utilization rate is higher in the recombinant yeast strain than in the wild type strain. Even with the same initial cell density (OD₆₀₀), the recombinant yeast strain consumed all galactose in about 14 hours, and produced 18.7 g/L of ethanol. In contrast, the wild type strain produced 17.8 g/L of ethanol and required 26 hours to consume all galactose. This indicates that the volumetric productivity of ethanol increases from 0.68 g/L per hour determined for the wild-type strain to 1.33 g/L per hour for the recombinant strain. That is, the volumetric productivity of ethanol increases by 95%, when the yeast strain was transformed with the gene encoding truncated TUP1 protein.

### EXAMPLE 3

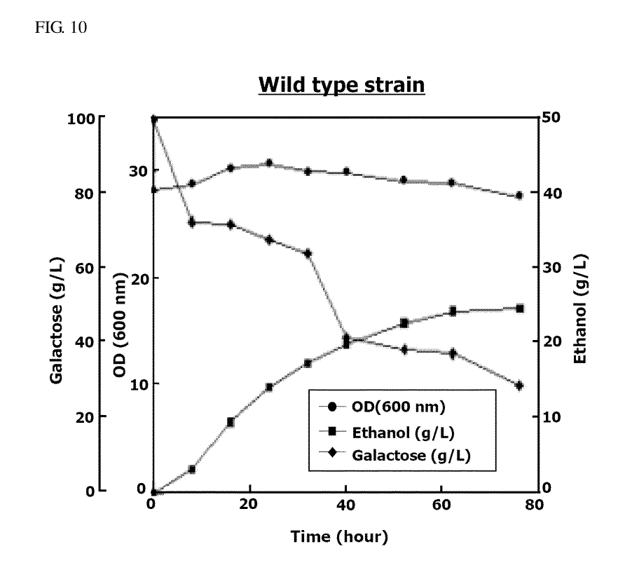

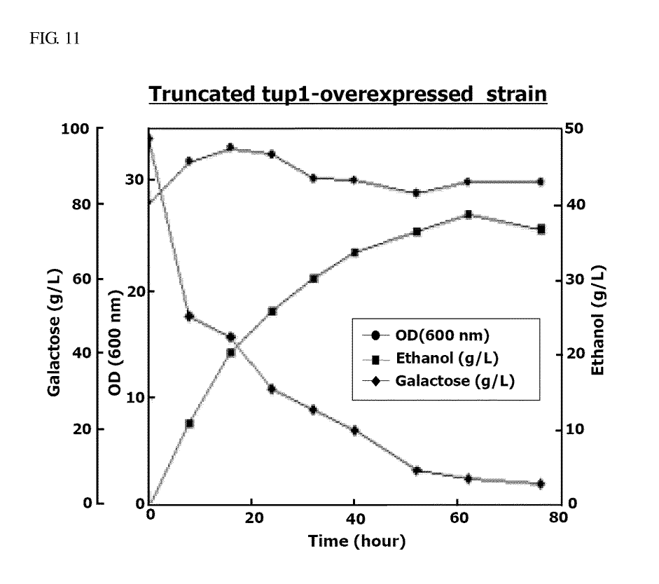

The wild type yeast strain (wild type) was compared to the recombinant yeast strain transformed with the gene encoding truncated TUP1 protein (truncated tup1) prepared in Preparation Example 1 with respect to galactose utilization rate and ethanol volume productivity by the same method as described in Example 1, except that the yeast strains were cultured in minimal medium containing only 10% galactose as a carbon source for 80 hours. The results are shown in FIGS. 10 and 11, for wild-type and recombinant yeast strains, respectively, and Table 3.

TABLE 3

| | Wild type | Truncated tup1 |
|---|---|---|
| Galactose consumption time | 80 h | 80 h |
| Ethanol concentration | 25 g/l | 38 g/l |
| Volumetric productivity | 0.31 g/lh | 0.48 g/lh |

The results show that galactose utilization is higher in the recombinant yeast strain than in the wild type strain. Even with the same initial cell density, the recombinant yeast strain left only 5 g/L of galactose after an 80-hour fermentation and produced 38 g/L of ethanol. In contrast, the wild type strain left 23 g/L of galactose after fermentation for 80 hours and produced 25 g/L of ethanol.

### EXAMPLE 4

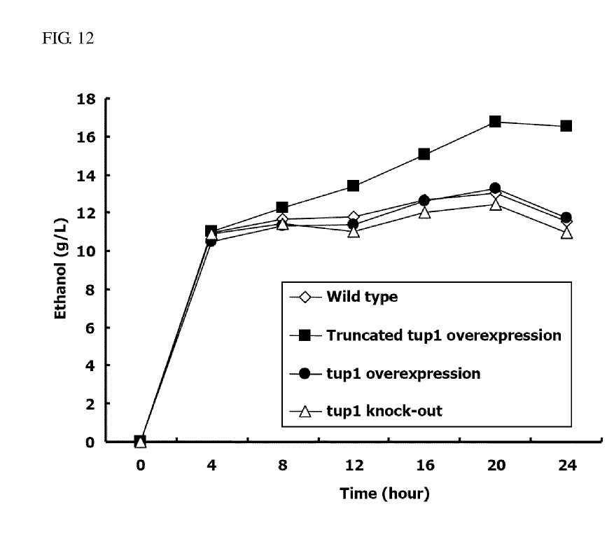

Volumetric productivity of ethanol was determined for a 1L culture of each of the following yeast strains: the yeast strain prepared in Preparative Example 1, overexpressing the gene encoding truncated TUP1 protein (truncated tup1 overexpression); a wild-type yeast strain (wt control), a yeast strain overexpressing the wild-type TUP1-gene (wt tup 1 overexpression) and a TUP1-knock-out yeast strain (tup1 knockout). Each strain was cultured in minimal medium containing a mixture of 2% each glucose and galactose, and the volumetric productivity of ethanol was determined. The results are shown in FIG. 12, and Table 4.

TABLE 4

| | | truncated tup1 | wt tup 1 | |
|---|---|---|---|---|
| Time (h) | wt control | overexpression | overexpression | tup1 knockout |
| 0 | 0 | 0 | 0 | 0 |
| 4 | 10.9586 | 11.024 | 10.4684 | 10.8903 |
| 8 | 11.665 | 12.2271 | 11.3107 | 11.3987 |
| 12 | 11.7704 | 13.4094 | 11.3433 | 11.0397 |
| 16 | 12.691 | 15.0325 | 12.6048 | 12.0053 |
| 20 | 13.0141 | 16.7613 | 13.29 | 12.4637 |
| 24 | 11.5268 | 16.5114 | 11.75 | 10.9448 |

The results show that volumetric productivity of ethanol by the the wild type yeast strain, the yeast strain overexpressin wild-type TUP1, and the TUP1-knock-out yeast strain was similar in the initial presence of 2% galactose/2% glucose. However, the yeast strain overexpressing the gene encoding truncated TUP1 protein produced 16.5 g/L of ethanol in the 24-hour fermentation. Accordingly, volumetric productivity of ethanol was higher for the yeast strain overexpressing the gene encoding truncated TUP1 protein than for the wild-type control yeast strain.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While exemplary embodiments have been disclosed herein, it should be understood that various modifications or changes to the exemplary embodiments may be possible. Such modifications or changes are not to be regarded as a departure from the spirit and scope of the present application, and all such modifications as would be obvious to one skilled in the art are intended to be included within the scope of the following claims.

What is claimed is:

1. An isolated polynucleotide comprising a nucleotide sequence encoding a polypeptide, the amino acid sequence of which consists of SEQ ID NO:2.

2. The isolated polynucleotide of claim 1, wherein the nucleotide sequence consists of SEQ ID NO:1.

3. An isolated polypeptide, the amino acid sequence of which consists of SEQ ID NO:2.

4. A recombinant vector comprising the isolated polynucleotide of claim 1.

5. The recombinant vector of claim 4, which is a plasmid.

6. The recombinant vector of claim 4, wherein the plasmid comprises pRS424.

7. The recombinant vector of claim 4, wherein the isolated polynucleotide is operably linked to an expression control sequence.

8. The recombinant vector of claim 4, wherein the isolated polynucleotide consists of SEQ ID NO: 1.

9. A recombinant microorganism comprising the isolated polynucleotide of claim 1.

10. The recombinant microorganism of claim 9, wherein the isolated polynucleotide is operably linked to an expression control sequence.

11. The recombinant microorganism of claim 9, comprising a vector comprising the isolated polynucleotide.

12. The recombinant microorganism of claim 9, wherein the isolated polynucleotide consists of SEQ ID NO:1.

13. The recombinant microorganism of claim 9, which is a yeast.

14. The recombinant microorganism of claim 13, wherein the yeast is selected from yeasts of the genus Saccharomyces, yeasts of the genus Pachysole, yeasts of the genus Clavispora, yeasts of the genus Kluyveromyces, yeasts of the genus Debaryomyces, yeasts of the genus Schwanniomyces, yeasts of the genus Candida, yeasts of the genus Pichia, and yeasts of the genus Dekkera.

15. The recombinant microorganism of claim 14, wherein the yeast is Saccharomyces cerevisiae CEN.PK2-1D/pRS424-truncated TUP1 (Accession No. KCTC 11387 BP).

16. A method of producing ethanol from a galactose-containing carbon source comprising
culturing the recombinant microorganism of claim 9 in a galactose-containing carbon source such that ethanol is produced.

17. The method of claim 16, wherein production of ethanol is increased by overexpression of the isolated polynucleotide.

18. The method of claim 16, wherein ethanol production is increased by at least about 30% compared to ethanol production during culturing of the microorganism without overexpression of the isolated polynucleotide.

19. The method of claim 16, wherein the galactose-containing carbon source contains only galactose, or a mixture of glucose and galactose.

20. The method of claim 16, wherein the galactose-containing carbon source contains at least about 4% of galactose.

21. The method of claim 16, further comprising recovering the ethanol.

22. A method of producing a recombinant microorganism, transforming a microorganism with the recombinant vector of claim 4.

23. The method of claim 22, wherein catabolism of galactose by the transformed microorganism is greater than by the microorganism.

24. The method of claim 22, wherein ethanol production from a culture with galactose as a carbon source is greater for the transformed microorganism than for the microorganism.

25. The method of claim 22, wherein the microorganism is a yeast selected from yeasts of the genus Saccharomyces, yeasts of the genus Pachysole, yeasts of the genus Clavispora, yeasts of the genus Kluyveromyces, yeasts of the genus Debaryomyces, yeasts of the genus Schwanniomyces, yeasts of the genus Candida, or yeasts of the genus Pichia, and yeasts of the genus Dekkera.

26. The method of claim 25, wherein the microorganism is a yeast of the genus Saccharomyces.

27. The method of claim 22, wherein the isolated polynucleotide consists of SEQ ID NO:1.


SEQUENCE LISTING (2 sequences)

SEQ ID NO: 1 - DNA, 855 bp, S. cerevisiae
  truncated tup1 gene
atgactgccagcgtttcgaatacgcagaataagctgaatgagcttctcgatgccatcagacaggagtttctccaagtctcacaagaggcaaatacctaccgtcttcaaaaccaaaaggattacgatttcaaaatgaaccagcagctggctgagatgcagcagataagaaacaccgtctacgaactggaactaactcacaggaaaatgaaggacgcgtacgaagaagagatcaagcacttgaaactagggctggagcaaagagaccatcaaattgcatctttgaccgtccagcaacagcggcaacagcaacagcagcaacaggtccagcagcatttacaacagcaacagcagcagctagccgctgcatctgcatctgttccagttgcgcaacaaccaccggctactacttcggccaccgccactccagcagcaaacacaactactggttcgccatcggccttcccagtacaagctagccgtcctaatctggttggctcacagttgcctaccaccactttgcctgtggtgtcctcaaacgcccaacaacaactaccacaacagcaactgcaacagcagcaacttcaacaacagcaaccacctccccaggtttccgtggcaccattgagtaacacagccatcaacggatctcctacttctaaagagaccactactttaccctctgtcaaggcacctgaatctacgttgaaagaaactgaaccggaaaataataatacctcgaagataaatgacaccggatccgccaccacggccaccactaccaccgcaactgaaactgaaatcaaacctaaggaggaagacgccaccccggctagtttgcaccaggatcactactta

SEQ ID NO: 2 - protein, 284 aa, S. cerevisiae
  truncated TUP1 protein
MTASVSNTQNKLNELLDAIRQEFLQVSQEANTYRLQNQKDYDFKMNQQLAEMQQIRNTVYELELTHRKMKDAYEEEIKHLKLGLEQRDHQIASLTVQQQRQQQQQQQVQQHLQQQQQQLAAASASVPVAQQPPATTSATATPAANTTTGSPSAFPVQASRPNLVGSQLPTTTLPVVSSNAQQQLPQQQLQQQQLQQQQPPPQVSVAPLSNTAINGSPTSKETTTLPSVKAPESTLKETEPENNNTSKINDTGSATTATTTTATETEIKPKEEDATPASLHQDHY